(19) United States
(12) Patent Application Publication
(10) Pub. No.: US 2020/0135315 A1
(43) Pub. Date: Apr. 30, 2020

(54) Unsaleable Pharmaceutical Item Handling and Disposal Systems, Methods, and Devices

(71) Applicant: Waste Repurposing International, Inc., Austin, TX (US)

(72) Inventors: Christopher B. Ripley, Lichtfield, CT (US); Charles Bartol Vallely, Austin, TX (US)

(73) Assignee: Waste Repurposing International, Inc., Austin, TX (US)

(21) Appl. No.: 16/176,691

(22) Filed: Oct. 31, 2018

(51) Int. Cl. G16H 20/10; G06Q 10/00; G16H 40/20

(52) U.S. Cl. CPC G16H 20/10; G16H 40/20; G06Q 10/30

(57) ABSTRACT

In some embodiments, a device may include at least one camera, a display device, and a processor coupled to the camera and the display device. The camera may be configured to capture first optical data of a unsaleable pharmaceutical item and second optical data of an operator. The processor may be configured to provide a graphical interface to the display device. The graphical interface may include information to guide the operator through a process of sorting unsaleable pharmaceutical items for disposal, the graphical interface including the first optical data and the second optical data.

### FIELD

The present disclosure is generally related to unsaleable pharmaceutical items, and more particularly to systems, methods, and devices configured to manage handling and disposal of unsaleable pharmaceutical items.

### BACKGROUND

The Environmental Protection Agency (EPA) has imposed regulations for safe disposal of unsaleable pharmaceutical items. Unsaleable pharmaceutical items may include intravenous (IV) preparations, general compounding, spills, breakages, partially used vials, syringes, IVs, discontinued preparations, unused preparations, unused unit dose repacks, patients' personal medications, outdated pharmaceuticals, other items, or any combination thereof. Additionally, the unsaleable pharmaceutical items can include returns of unused prescription medications and over-the-counter health products.

Under existing EPA regulations and until a new rule is finalized and adopted, healthcare facilities and other business entities that generated or received unsaleable pharmaceutical items are required to manage such waste items according to the hazardous waste generator requirements, which are governed under the Resource Conservation and Recovery Act (RCRA) standards that can be found in Title 40 of the Code of Federal Regulations (CFR) in part 262. The hazardous waste generator requirements or regulations are designed to that the waste items are appropriately identified and handled safely to protect human health and the environment, while minimizing interference with daily business operations.

Unfortunately, pharmacists, nurses, and other employees of waste generators often do not receive training on pharmaceutical waste or unsaleable pharmaceutical item management during their academic studies, and safety or compliance managers may be unfamiliar with the active ingredients and formulations of pharmaceutical compounds. Such unfamiliarity can expose a waste generator or an entity that is tasked with disposing of unsaleable pharmaceutical items to serious violations and large penalties. In an example, frequently used pharmaceuticals, such as physostigmine, warfarin, and nine chemotherapeutic agents, are regulated as hazardous waste under the RCRA, and failure to comply with hazardous waste regulations in the handling and disposal of such pharmaceuticals can result in serious violations and large penalties.

### SUMMARY

Embodiments of systems, methods, and devices are described below that may be configured to guide an operator, such as an untrained employee of a waste generator or an entity that is tasked with disposing of unsaleable pharmaceutical items, through a process of identifying, weight, labeling, and packaging unsaleable pharmaceutical items. In an example, a device may be configured to process unsaleable pharmaceutical items one at a time.

In some embodiments, a device may be configured to provide a graphical interface including instructions and including data determined from optical data corresponding to the unsaleable pharmaceutical item to a display. The instructions may guide the operator through a plurality of steps for quantifying and safely storing the unsaleable pharmaceutical items. The device may store data related to the unsaleable pharmaceutical items in a memory, and the operator may place the processed unsaleable pharmaceutical item in a storage bin. When the storage bin is full or when it is time for collection of the unsaleable pharmaceuticals, the device may provide a graphical interface to the display prompting and guiding the operator through a process of packaging the unsaleable pharmaceuticals for shipping and may print manifests and shipping labels to facilitate the packaging and shipping process.

In some embodiments, the device may capture video of the operator while the operator is processing unsaleable pharmaceutical items, and images from the video may be stored with the unsaleable pharmaceutical item record in the memory, providing an audit trail and chain-of-custody for each waste item. Further, the device may verify the unsaleable pharmaceutical items are placed in the shipping container by weight, ensuring that a total weight of the processed unsaleable pharmaceutical items removed from the bin to be placed in the shipping packaging matches the total weight of the unsaleable pharmaceutical items in the shipping container. Other embodiments are also possible.

In some embodiments, a system may include a device that can guide an operator to scan and weigh a plurality of unsaleable pharmaceutical containers, one at a time, and to automatically store an identifier associated with the scanned waste item and its corresponding weight in a memory. The systems, methods, and devices may further include a camera to capture optical data associated with the operator and optical data associated with the unsaleable pharmaceutical item to provide a video audit of the disposal efforts. The systems, methods, and devices may also include a display, which may present a graphical interface including data and instructions and including one or more user-selectable elements accessible by the operator to record data, update data records, and optical data of the operator and of the unsaleable pharmaceutical item.

In some embodiments, when it is time for waste pickup by an authorized pharmaceutical disposal vendor, the operator may interact with one or more of the user-selectable elements within the graphical interface to create a package for shipment, to weigh the package container, to verify the weight and label of each item placed into the package container, to verify a final weight, to print a manifest, and to print a shipping label for shipment so that the pharmaceutical disposal vendor can collect the shipping container for transport. The printed manifest can match a digital manifest stored by the sorter device, and optionally provided to the pharmaceutical identification system, which may be configured to track shipments and manifests from origination to disposal. The contents can be verified by weight, the container sealed, and the scanned waste items assigned to the package so that

In some embodiments, a device may include at least one camera, a display device, and a processor coupled to the camera and the display device. The camera may be configured to capture first optical data of a unsaleable pharmaceutical item and second optical data of an operator. The processor may be configured to provide a graphical interface to the display device. The graphical interface may include information to guide the operator through a process of sorting unsaleable pharmaceutical items for disposal, and may include the first optical data and the second optical data.

In other embodiments, a device may include at least one optical sensor, a display device, and a processor coupled to the at least one optical sensor and the display device. The optical sensor may be configured to capture first optical data associated with a unsaleable pharmaceutical product and to capture second optical data associated with an operator. The processor may be configured to determine a disposal bin from a plurality of disposal bins based on the first optical data and provide a graphical interface including a bin identifier, the first optical data, and the second optical data to the display device.

In still other embodiments, a method may include receiving first optical data of a unsaleable pharmaceutical item from at least one camera of a unsaleable pharmaceutical sorter device. The method may further include receiving second optical data of an operator from the at least one camera. Additionally, the method can include providing a graphical interface to a display device of the unsaleable pharmaceutical sorter device. The graphical interface may include information to guide the operator through a process of sorting unsaleable pharmaceutical items for disposal. The graphical interface may also include the first optical data and the second optical data.

### BRIEF DESCRIPTION OF THE DRAWINGS

FIG. 1 depicts a diagram of a system configured to facilitate handling and disposal of unsaleable pharmaceutical items, in accordance with certain embodiments of the present disclosure.

FIG. 2 depicts a block diagram of a system configured to facilitate handling and disposal of unsaleable pharmaceutical items, in accordance with certain embodiments of the present disclosure.

FIG. 3 depicts a method of handling unsaleable pharmaceutical items, in accordance with certain embodiments of the present disclosure.

FIG. 4 depicts a method of handling and packaging of unsaleable pharmaceutical items for transport and disposal, in accordance with certain embodiments of the present disclosure.

FIG. 5 depicts a graphical interface provided to a display of the system of FIGS. 1 and 2 prior to authentication of the user, in accordance with certain embodiments of the present disclosure.

FIG. 6 depicts a graphical interface provided to the display of the system of FIGS. 1 and 2 for selecting a user from a list of users as part of an authentication process, in accordance with certain embodiments of the present disclosure.

FIG. 7 depicts a login page of the graphical interface of FIG. 6 to receive a password, in accordance with certain embodiments of the present disclosure.

FIG. 8 depicts a graphical interface provided to the display of the system of FIG. 1 prior to scanning a first unsaleable pharmaceutical product using the system of FIGS. 1 and 2, in accordance with certain embodiments of the present disclosure.

FIG. 9 depicts a graphical interface provided to the display of the system of FIGS. 1 and 2 after scanning a selected unsaleable pharmaceutical product and accessible by the operator to specify a size of the container, in accordance with certain embodiments of the present disclosure.

FIG. 10 depicts a graphical interface provided to the display of the system of FIGS. 1 and 2 after scanning and identifying the container size, in accordance with certain embodiments of the present disclosure.

FIG. 11 depicts the graphical interface of FIG. 10 after applying a label to seal and obscure a label of the container, in accordance with certain embodiments of the present disclosure.

FIG. 12 depicts a graphical interface provided to the display to the system of FIGS. 1 and 2 for handling an empty container, in accordance with certain embodiments of the present disclosure.

FIG. 13 depicts the graphical interface provided to the display of the system of FIGS. 1 and 2 after confirming that the bottle is empty using the interface of FIG. 12, in accordance with certain embodiments of the present disclosure.

FIG. 14 depicts the graphical interface of FIG. 13 after applying a label to seal and obscure a label of the container, in accordance with certain embodiments of the present disclosure.

FIG. 15 depicts a graphical interface provided to the display of the system of FIGS. 1 and 2 after some unsaleable pharmaceutical items have been scanned and while awaiting a next scan, in accordance with certain embodiments of the present disclosure.

FIG. 16 depicts a graphical interface provided to the display of the system of FIGS. 1 and 2 depicting a list of waste bins and data related to the contents of each bin, in accordance with certain embodiments of the present disclosure.

FIG. 17 depicts a graphical interface provided to the display of the system of FIGS. 1 and 2 depicting a selected waste bin and an empty shipping container, in accordance with certain embodiments of the present disclosure.

FIG. 18 depicts the graphical interface of FIG. 17 including a weight of the empty shipping container, in accordance with certain embodiments of the present disclosure.

FIG. 19 depicts a graphical interface provided to the display of the system of FIGS. 1 and 2 configured to guide an operator through a process of transferring unsaleable pharmaceutical items from a selected bin to the shipping container, showing that no items have yet been transferred, in accordance with certain embodiments of the present disclosure.

FIG. 20 depicts the graphical interface of FIG. 19 with one item transferred to the shipping container, in accordance with certain embodiments of the present disclosure.

FIG. 21 depicts a graphical interface provided to the display of the system of FIG. 1 configured to guide the operator to seal the shipping container, in accordance with certain embodiments of the present disclosure.

FIG. 22 depicts the graphical interface of FIG. 16 with the “Controls” bin reset to reflect zero items, in accordance with certain embodiments of the present disclosure.

FIG. 23 depicts a graphical interface provided to the display of the device of FIG. 1 including a list of shipping containers or packages, in accordance with certain embodiments of the present disclosure.

In the following discussion, the same reference numbers are used in the various embodiments to indicate the same or similar elements.

### DETAILED DESCRIPTION OF ILLUSTRATIVE EMBODIMENTS

Embodiments of systems, methods and devices described below are configured to provide secure, traceable, and efficient disposal of unsaleable pharmaceutical items. In some embodiments, a device may be configured to capture optical data, including an identifier associated with a unsaleable pharmaceutical item, and to print an adhesive label configured to provide a tamper-proof seal for the unsaleable pharmaceutical container and to obfuscate information on a label of the container to ensure that private information remains protected. The device may be configured to track all information associated with the item as it is identified and stored in a storage bin, and subsequently when it is packaged into a shipping container and collected by a licensed vendor.

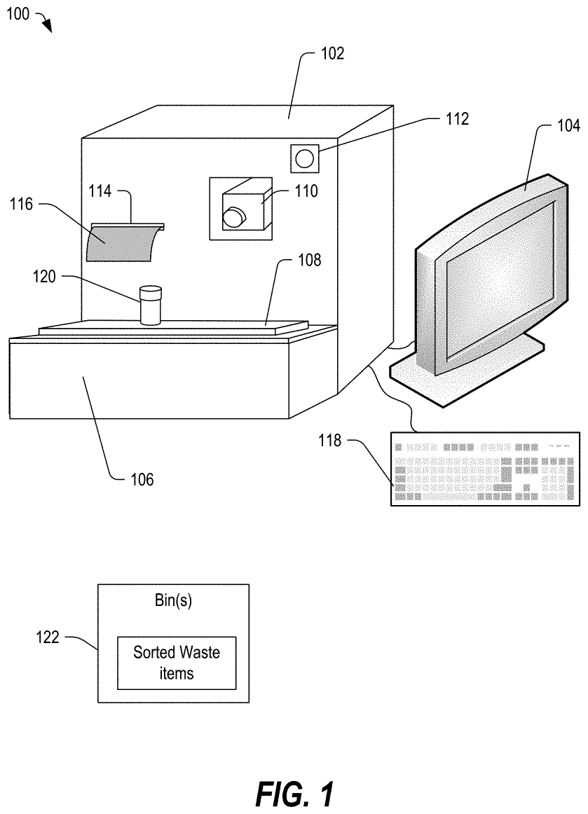

In some embodiments, the device may be configured to acquire images of the discarded pharmaceuticals and to acquire video of the operator throughout the workflow. The device may utilize a barcode reader and other optical sensors to capture the data, may utilize the data to identify the waste product and to identify an appropriate bin of a plurality of bins for the waste product. The device may save pharmacists time by automating the data collection process and may help pharmacists avoid costly mistakes by ensuring proper identification and categorization of the unsaleable pharmaceutical items, avoiding costly mistakes. One possible embodiment of a system configured to facilitate handling and disposal of unsaleable pharmaceutical items is described below with respect to FIG. 1.

FIG. 1 depicts a diagram of a system 100 configured to facilitate handling and disposal of unsaleable pharmaceutical items, in accordance with certain embodiments of the present disclosure. The system 100 may include a unsaleable pharmaceutical sorter device 102 coupled to a display device 104. The unsaleable pharmaceutical sorter device 102 may include a base portion 106 configured to support a scale 108. The unsaleable pharmaceutical sorter device 102 may further include a first optical sensor (camera, barcode reader, or other image capture device) 110 configured to capture data related to a unsaleable pharmaceutical item 120. The unsaleable pharmaceutical sorter device 102 may further include a camera 112 configured to capture video or images of an operator of the unsaleable pharmaceutical sorter device 102 during operation. In some embodiments, instead of the item camera 110 and the operator camera 112, the unsaleable pharmaceutical sorter device 102 may include a single camera or optical sensor configured to capture optical data associated with both the unsaleable pharmaceutical item 120 and the operator. Other embodiments are also possible.

The unsaleable pharmaceutical sorter device 102 may further include a printer, generally indicated at 114, configured to print a document 116, such as an adhesive label, a manifest, or any combination thereof. The unsaleable pharmaceutical sorter device 102 may also include one or more input devices 118, such as a keyboard, a mouse, a pointer device, other input elements, or any combination thereof.

In operation, an operator may interact with the system 100 to sort unsaleable pharmaceutical items 120 into one or more bins 122, which may store the unsaleable pharmaceutical items 120 until they are ready to be packaged for transport and disposal. The unsaleable pharmaceutical sorter device 102 may provide a graphical interface to the display device 104 prompting the operator to select a unsaleable pharmaceutical item 120 and to place the unsaleable pharmaceutical item 120 onto the scale 108. The scale 108 may capture a weight of the unsaleable pharmaceutical item 120 on the scale 108 while concurrently capturing optical data via the optical sensor 110. In some embodiments, the scale 108 may be sufficiently sensitive to detect a weight of a single pill.

While the optical sensor 110 captures optical data, the camera 112 may capture video of the operator. In some embodiments, the camera 112 may be configured to capture video continuously and to provide the video to a buffer, making it possible to selectively store the captured video in memory when the operator is interacting with the unsaleable pharmaceutical sorter device 102, while discarding the video when the device is not in use. Upon detection of a weight on the scale 108, a processor of the unsaleable pharmaceutical sorter device 102 may store video from the buffer into memory. In an example, the unsaleable pharmaceutical sorter device 102 may store the video data surrounding the placement and handling of the unsaleable pharmaceutical item 120, such as beginning a period of time prior to detection of the unsaleable pharmaceutical item 120 on the scale 108 until a period of time after the unsaleable pharmaceutical item 102 has been moved from the scale 108. The video may capture images of the operator as he or she handles the unsaleable pharmaceutical item 120.

In an embodiment, the unsaleable pharmaceutical sorter device 102 may be configured to identify the unsaleable pharmaceutical item 120 based on the optical data and may determine a bin from a plurality of bins for storage and disposal of the unsaleable pharmaceutical item 120 by comparing the identified waste item to a set of handling and disposal rules. The unsaleable pharmaceutical sorter device 102 may print an adhesive label to seal the container closed and to obscure any existing label on the container. The graphical interface may instruct the operator to apply the adhesive label to the unsaleable pharmaceutical item 120 and to place the waste item into the selected bin. The unsaleable pharmaceutical sorter device 102 may store data related to the unsaleable pharmaceutical item 120 in memory together with an identifier associated with the bin so that the disposal can be included in a manifest and subsequently tracked.

In some embodiments, each unsaleable pharmaceutical item 120 can be weighed before and after application of the adhesive label that seals and obscures the existing label. The total weight of each of the unsaleable pharmaceutical items 120 stored in each bin. When a particular bin is full or when it is time for pickup of the unsaleable pharmaceutical items, the unsaleable pharmaceutical sorter device 102 may provide a graphical interface to the display device 104 prompting the operator to weigh a shipping container and then to relocate each unsaleable pharmaceutical item 120 from its bin to the shipping container, monitoring the changes to verify that the total weight of the contents of the shipping container. Further, the unsaleable pharmaceutical sorter device 102 may print a first document (a manifest of unsaleable pharmaceutical items in the shipping container) and a second document (a shipping label). Other embodiments are also possible.

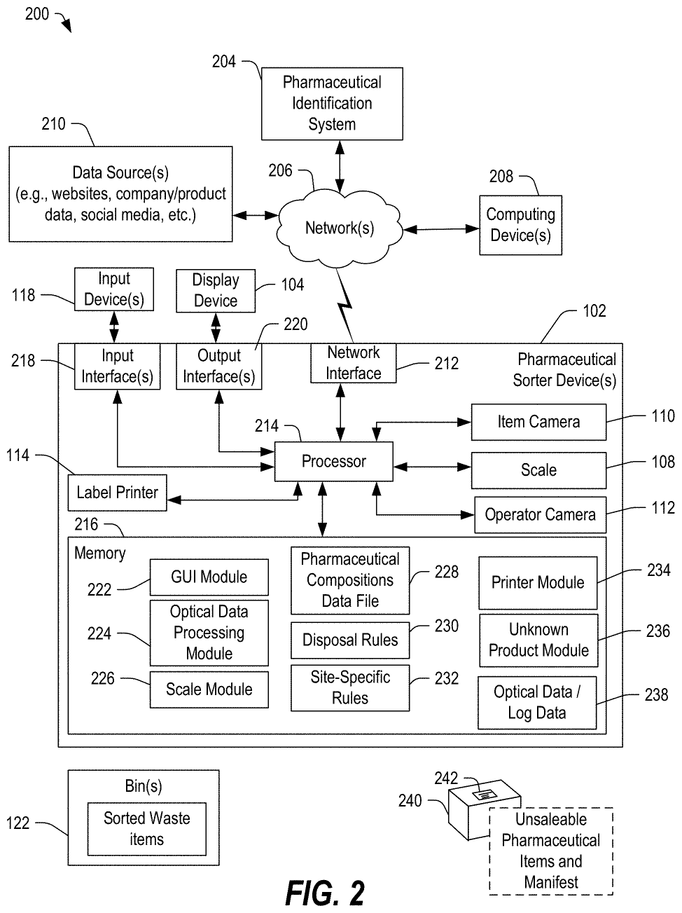

The unsaleable pharmaceutical sorter device 102 may store records corresponding to each shipping container, and optionally may communicate data related to each shipping container to a third party, enabling for third-party audits. One possible example of a system including the unsaleable pharmaceutical sorter device 102 configured to communicate with a third party system is described below with respect to FIG. 2.

FIG. 2 depicts a block diagram of a system 200 configured to facilitate handling and disposal of unsaleable pharmaceutical items, in accordance with certain embodiments of the present disclosure. The system 200 may be an embodiment of the system 100 of FIG. 1. In this example, the system 200 may the unsaleable pharmaceutical sorter device 102, which may be coupled to a display device 104 and to one or more input devices 118. Further, the unsaleable pharmaceutical sorter device 102 may be configured to communicate with a unsaleable pharmaceutical identification system 204, one or more computing devices 208, and one or more data sources 210 through a network 206, such as the Internet.

The unsaleable pharmaceutical sorter device 102 may include a network interface 212 configured to communicate with the network 206. The unsaleable pharmaceutical sorter device 102 may further include a processor 214 coupled to the network interface 212. The unsaleable pharmaceutical sorter device 102 may further include a memory 216 coupled to the processor 214. The unsaleable pharmaceutical sorter device 102 can include one or more input interfaces 218 configured to couple to the input devices 118 and one or more output interfaces 220, at least one of which may be coupled to the display device 104. The processor 214 may be coupled to the input interfaces 218 and to the output interfaces 220.

The unsaleable pharmaceutical sorter device 102 may further include an item camera 110 coupled to the processor 214 and configured to capture optical data associated with a unsaleable pharmaceutical item 120. The unsaleable pharmaceutical sorter device 102 may further include or be coupled to a scale 108 that is coupled to the processor 214 and configured to measure a weight of a unsaleable pharmaceutical item 120. The unsaleable pharmaceutical sorter device 102 may also include an operator camera 112 coupled to the processor 214 and configured (oriented or otherwise arranged) to capture video data of a view area in front of the unsaleable pharmaceutical sorter device 102 in order to video the operator during scanning operations.

The memory 216 may include a graphical user interface (GUI) module 222 that, when executed, may cause the processor 214 to generate a graphical interface and to provide the graphical interface to a display de vice 104. The graphical interface may include instructions or prompts to guide a user through a process of handling and packaging unsaleable pharmaceutical items. Further, the graphical interface may include data related to a particular unsaleable pharmaceutical item 120, data related to previously scanned waste items, user-selectable elements (such as buttons, links, tabs, text fields, other elements, or any combination thereof).

The memory 216 may also include an optical data processing module 224 that, when executed, may cause the processor 214 to extract barcode data or other information from the optical data captured by the item camera 110. The optical data processing module 224 may be configured to search a pharmaceutical compositions data file 228 based on the extracted data to identify the unsaleable pharmaceutical item 120. Further, the optical data processing module 224 may cause the processor 214 to determine disposal rules and procedures for the identified unsaleable pharmaceutical item 120. The optical data processing module 224 may also cause the processor 214 to determine site-specific rules 232, which may specify site-specific bin names or other information for the handling of the unsaleable pharmaceutical item 120. Further, the memory 216 can include a scale module 226 that, when executed, may cause the processor 214 to control the scale 108 to determine the weight of the unsaleable pharmaceutical item 120 and to process the weight based on data determined about the unsaleable pharmaceutical item 120 by the optical data processing module 224 to determine a quantity of pills or a volume of liquid.

The memory 216 can include a printer module 234 that, when executed, may cause the processor 214 to control the label printer 114 to print a manifest, a shipping label, a label to seal and obscure an unsaleable pharmaceutical item 120, or any combination thereof. The memory 216 can further include an unknown product module 236, which may be configured to search an inventory system (not shown) or to communicate with the unsaleable pharmaceutical identification system 204 to determine pharmaceutical composition data. The memory 216 can also include an optical data/log data file 238. The processor 214 may be configured to log each scan, the data corresponding to the identified unsaleable pharmaceutical product (including optical data, weight data, and quantity data), video data of the operator, a date/time stamp, bin assignment information, other data, or any combination thereof.

In the illustrated example, the operator may interact with a plurality of bins 122, each of which may store a particular class of unsaleable pharmaceutical item 120. Further, the operator may interact with (assemble) a plurality of shipping containers. One such shipping container 240 is shown that is labeled for transport using a shipping label 242 printed by the label printer 114. The sealed shipping container 240 may include one or more unsaleable pharmaceutical items 120 and an associated shipping manifest. Other embodiments are also possible.

In operation, as each unsaleable pharmaceutical item 120 is placed on the scale 108, the unsaleable pharmaceutical sorter device 102 may cause the item camera 110 and the operator camera 112 to capture optical data associated with the unsaleable pharmaceutical item 120 and the operator. The unsaleable pharmaceutical sorter device 102 may determine information about the unsaleable pharmaceutical item 120 (e.g., identify and weigh the item), print a label to seal and obscure the unsaleable pharmaceutical item 120, and provide a graphical interface including a bin identifier to prompt the operator to place the unsaleable pharmaceutical item 120 into a selected bin of a plurality of bins. Further, the unsaleable pharmaceutical sorter device 120 may update the optical data/log data 238 with the determined information, a date/time stamp, and the bin assignment.

Subsequently, the unsaleable pharmaceutical sorter device 102 may prompt the operator to package the unsaleable pharmaceutical items 120 from a particular bin into a shipping container. The unsaleable pharmaceutical sorter device 102 may be configured to print a manifest, print a shipping label, and update the optical data/log data 238 to associate selected unsaleable pharmaceutical items 120 with a package identifier and with shipping information so that the disposal of the unsaleable pharmaceutical can be quantified and tracked. Other embodiments are also possible.

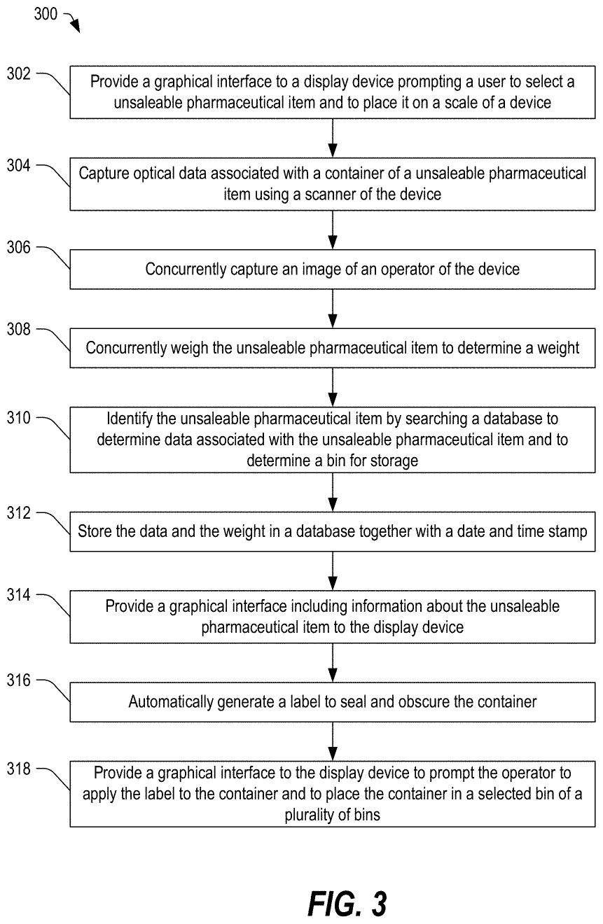

FIG. 3 depicts a method 300 of handling unsaleable pharmaceutical items, in accordance with certain embodiments of the present disclosure. At 302, the method 300 can include providing a graphical interface to a display device prompting a user to select an unsaleable pharmaceutical item and to place it on a scale of a device. It should be appreciated that the graphical interface may be presented to the display device 104 of the unsaleable pharmaceutical sorter device 102 and may include text prompting the operator to select a waste item, scan it, and weigh it.

At 304, the method 300 can include capturing optical data associated with a container of the unsaleable pharmaceutical item using a scanner of the device. In some embodiments, the scanner may include a camera configured to capture optical data associated with the container to detect a barcode or other identifier. In some embodiments, the scanner may include a barcode reader wand that can be controlled by the user to scan the product. Other embodiments are also possible.

At 306, the method 300 may include concurrently capturing an image of the operator of the device. In some embodiments, capturing an image may include capturing video data associated with the operator. The capturing of video data of the operator provides a level of authentication and verification, and may inspire a greater degree of attention to detail by the operators. Moreover, to the extent that a particular operator may be tempted to steal some of the unsaleable pharmaceutical items for resale and personal gain, the capturing of the video data may operate as a deterrent. Further, the video can be part of a larger audit trail, which can trace the handling of the unsaleable pharmaceutical items from receipt to disposal.

At 308, the method 300 can include concurrently weighing the unsaleable pharmaceutical item to determine a weight. In some embodiments, the scale 108 may be sufficiently accurate to detect a weight of a single pill, such that a change to the number of pills in a pill bottle can be determined based on a change in the weight. Other embodiments are also possible.

At 310, the method 300 can include identifying the unsaleable pharmaceutical item by searching a database to determine data associated with the unsaleable pharmaceutical item and to determine a bin for storage. In some embodiments, the unsaleable pharmaceutical items may be classified into different categories based on their respective chemical compositions. Each bin may store a different class of waste items, such as “Controls”, “non-controls”, “Warfarin bottles”, other categories, or any combination thereof. Once classified, the unsaleable pharmaceutical sorter device 102 may provide information within a graphical interface to indicate a bin for storage of a selected unsaleable pharmaceutical item.

At 312, the method 300 can include storing the data and weight in a database together with a date and time stamp. The stored data (in optical data/log data 238) may be used to track the handling and disposal of unsaleable pharmaceutical items and may be selected for printing of a manifest and for tracking disposal. Other embodiments are also possible.

At 314, the method 300 may include providing a graphical interface including information about the unsaleable pharmaceutical item to the display device. The information may include identification information, weight information, and a bin identifier.

At 316, the method 300 may include automatically generating a label to seal and obscure the container. The label may be printed with a barcode or other identifier that may obscure the content of the container while sealing the container closed.

At 318, the method 300 can include providing a graphical interface to the display device to prompt the operator to apply the label to the container and to place the container in a selected bin of a plurality of bins. Each bin may be labeled according to the content of the bin.

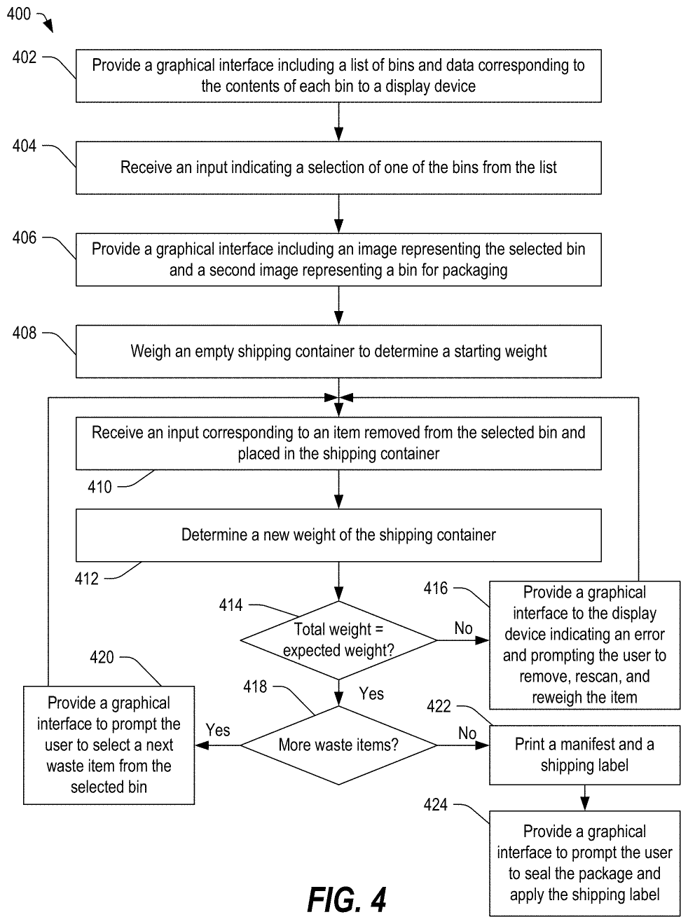

In the illustrated example, the blocks 302-318 may be combined and some of the blocks may be omitted, depending on the implementation. Further, additional steps or blocks may be included without departing from the scope of the disclosure. Further, it should be appreciated that the method 300 represents an embodiment of a process of scanning, identifying, classifying, and storing unsaleable pharmaceutical items. The method 300 may be repeated iteratively to store a plurality of unsaleable pharmaceutical items, each of which may be independently classified and stored in an appropriate bin with a sealing and obscuring label applied to its container until the unsaleable pharmaceutical item for a particular bin is repackaged into a shipping container for transport and disposal. One possible example of a method of packaging unsaleable pharmaceutical items for transport and disposal is described below with respect to FIG. 4.

FIG. 4 depicts a method 400 of handling and packaging of unsaleable pharmaceutical items for transport and disposal, in accordance with certain embodiments of the present disclosure. At 402, the method 400 may include providing a graphical interface including a list of bins and data corresponding to the contents of each bin to a display device. In some embodiments, the graphical interface may include a plurality of selectable elements, such as buttons, links, checkboxes, pull-down menus, tabs, other selectable elements, or any combination thereof. In one particular example, each bin of the list of bins may be presented within the graphical interface as a user-selectable button or link accessible by the operator to provide an input selecting one of the bins. At 404, the method 400 can include receiving an input indicating a selection of one of the bins from the list.

At 406, the method 400 can include providing a graphical interface including an image representing the selected bin and a second image representing a bin (or shipping container) for packaging. In some embodiments, the graphical interface may further include instructions prompting the operator to assemble or select a shipping container.

At 408, the method 400 can include weighing an empty shipping container to determine a starting weight (initial weight). At 410, the method 400 can include receiving an input corresponding to an item removed from the selected bin and placed in the shipping container. In some embodiments, the input may include an optical scan of the sealing and obscuring label of the unsaleable pharmaceutical item. It should be appreciated that the shipping container may be placed on the scale 108, and the unsaleable pharmaceutical item may be scanned and then placed into the shipping container on the scale 108.

At 412, the method 400 can include determining a new weight of the shipping container (including the newly added content). At 414, if the total weight of the shipping container does not equal the expected weight (i.e., the shipping container weight plus the weight of each newly added unsaleable pharmaceutical item), the method 400 may include providing a graphical interface to the display device indicating an error and prompting the user to remove, rescan, and reweigh the item, at 416. The method 400 may then return to 410 to rescan the unsaleable pharmaceutical item.

Otherwise, at 414, if the total weight equals the expected weight, the method 400 determines if there are more waste items to be sorted, at 418. If yes, the method 400 may include providing a graphical interface to prompt the user to select a next waste item from the selected bin, at 420. The method 400 returns to 410 to receive an input.

Returning to 418, if there are no more waste items, the method 400 can include printing a manifest and a shipping label, at 422. At 424, the method 400 may include providing a graphical interface to prompt the user to seal the package and apply the shipping label.

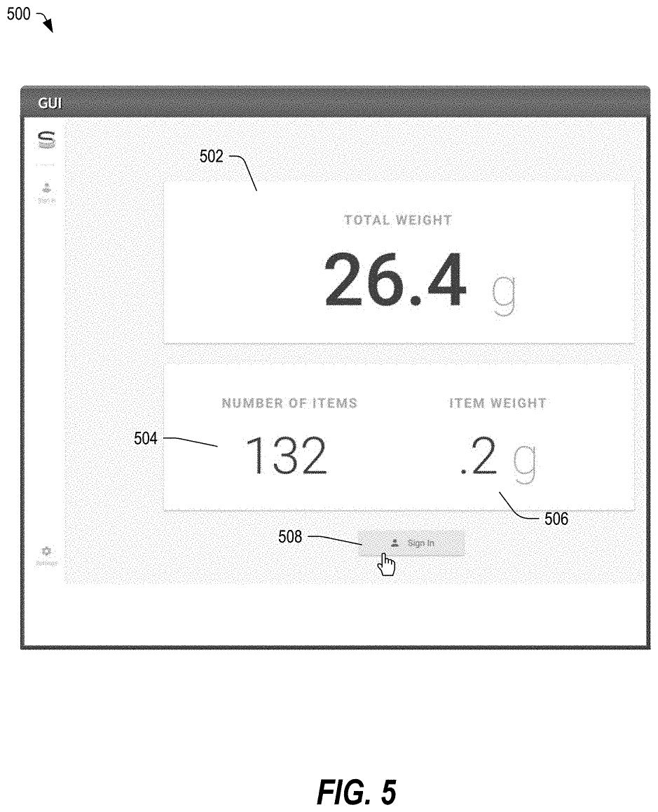

It should be appreciated that providing an interface in the method 400 of FIG. 4 or the method 500 of FIG. 5 may include updating data displayed within an interface. In some embodiments, providing an interface may include replacing a first interface with a second interface. Other embodiments are also possible.

FIG. 5 depicts a graphical interface 500 provided to a display device 104 of the system 100 of FIG. 1 or the system 200 of FIG. 2 prior to authentication of the user, in accordance with certain embodiments of the present disclosure. The graphical interface 500 can include a weight 502 of a unsaleable pharmaceutical item, a number of items 504 within a container of the unsaleable pharmaceutical item, and a weight 506 of the item. It may be appreciated that, from an idle state, the unsaleable pharmaceutical sorter device 102 may be activated (causing the device 102 to transition from an idle (low power) state to an active (operating power) state) in response to detecting a unsaleable pharmaceutical item 120 on the scale 108.

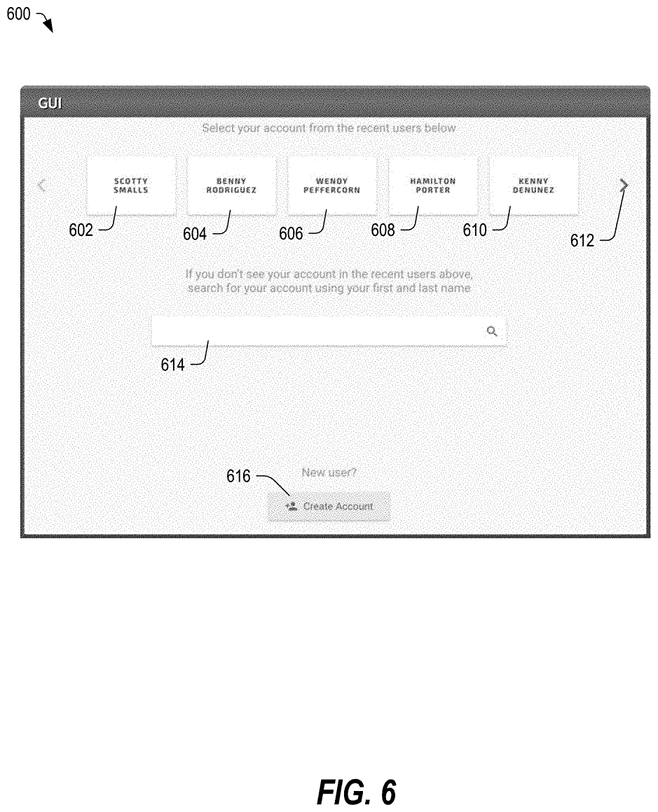

In the illustrated example, the graphical interface 500 includes a user-selectable option (“Sign In” button 508) accessible by an operator to initiate a login operation. The login operation may be desirable to identify the operator for the purpose of providing an audit trail as well as for accountability. One possible graphical interface that can be provided to the display device 104 in response to selection of the Sign In button 508 is described below with respect to FIG. 6.

FIG. 6 depicts a graphical interface 600 provided to the display device 104 of the unsaleable pharmaceutical sorter device 102 of FIGS. 1 and 2 for selecting a user from a list of users as part of an authentication process, in accordance with certain embodiments of the present disclosure. In the illustrated example, the graphical interface 600 may present a list of recent users as selectable buttons, such as a “Scotty Smalls” button 602, a “Benny Rodriguez” button 604, a “Wendy Peffercorn” button 606, a “Hamilton Porter” button 608, and a “Kenny Denunez” button 610. Further, the graphical interface 600 may include a selectable button or arrow 612 accessible by the operator to view other “recent users”. Selection of the arrow 612 may cause the row of buttons 602, 604, 606, 608, and 610 to shift left, admitting additional “recent” users as additional buttons.

The graphical interface 600 may further include a text field 614 accessible by the operator to search for a name of a registered user in memory. The graphical interface 600 may further include a button 616 labeled “Create Account”, which may be accessed by an operator to set up a new user. Other embodiments are also possible.

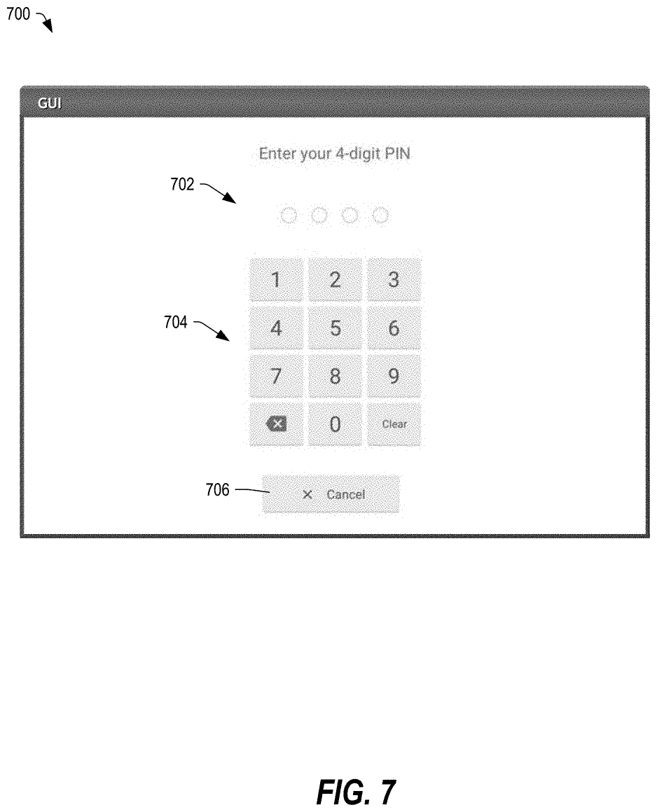

In response to selecting one of the registered users, the graphical interface 600 may change to provide user-selectable elements accessible by the operator to access an account. One possible example of an authentication page to receive a password or other credential is described below with respect to FIG. 7.

FIG. 7 depicts a login page 700 of the graphical interface 600 of FIG. 6 to receive a password, in accordance with certain embodiments of the present disclosure. The login page includes an indicator 702 configured to represent a number of characters entered or to be entered. The login page 700 further includes a plurality of selectable-elements 704, such as a numeric keypad, which may be accessed by an operator to enter a pin or password. The login page 700 may further include a “Cancel” button.

In some embodiments, if the operator enters an incorrect password for a selected operator, the graphical interface 600 may provide an indication of the incorrect password before returning to the graphical interface 600 to try again. Other embodiments are also possible.

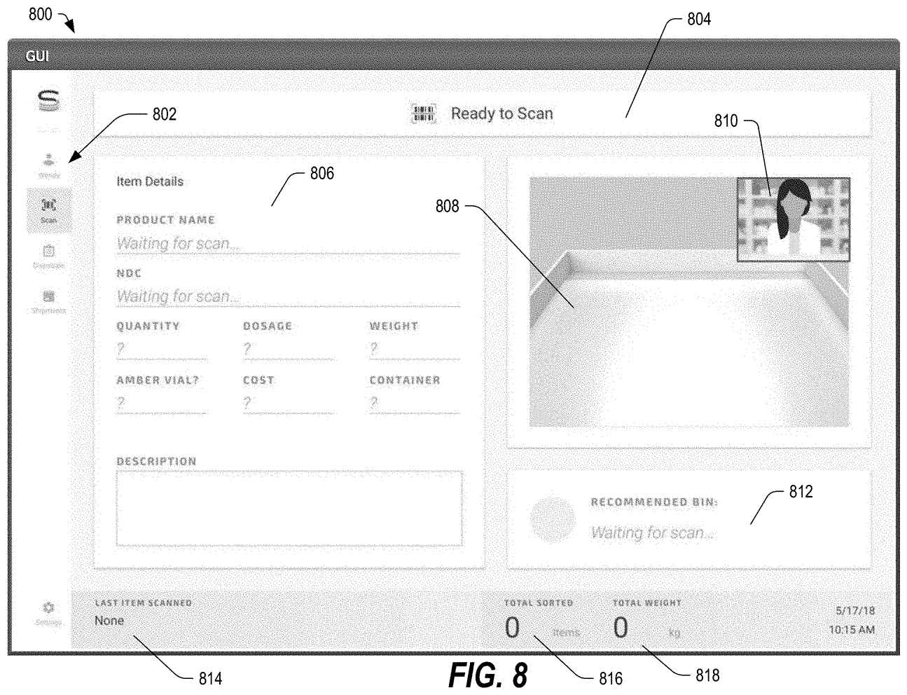

FIG. 8 depicts a graphical interface 800 provided to the display device 104 of the unsaleable pharmaceutical sorter device 102 of FIGS. 1 and 2 prior to scanning a first unsaleable pharmaceutical product, in accordance with certain embodiments of the present disclosure. The graphical interface 800 can include a plurality of selectable menu options 802, which may be accessible by an operator to select a “Home” view, a “Scan” view, a “Disposals” view, and a “Shipment” view. The Home view may present system information, operator information, and other information as well as options for configuring the device. The Disposals view may be accessible by the operator to view a list of bins and information about unsaleable pharmaceutical items within each bin. The Shipment view may be accessible by the operator to view a list of packages that have been sealed, labeled, and at least prepared for transport and disposal. Selecting one of the listed packages may allow the operator to view manifest information identifying the unsaleable pharmaceutical items included in the selected package (or shipping container).

In the illustrated example, the Scan view is selected, and the graphical interface 800 displays a scan status indicator 804, item details 806, and other information. In this instance, since no product has been scanned, the scan status indicator 804 includes the text “Ready to Scan.” The graphical interface 800 further includes a first image 808 from the item camera 110, and includes a second image 810 from the operator camera 112. The images 808 and 810 may represent video streams and may be presented (as shown) within the graphical interface 800 as a picture-in-picture implementation.

The graphical interface 800 may include a “Recommended Bin” indicator 812, which indicates that the status is “Waiting for Scan . . . ” Further, the graphical interface 800 can include a last item scanned 814, a total number of sorted items 816, and a total weight of the scanned items 818.

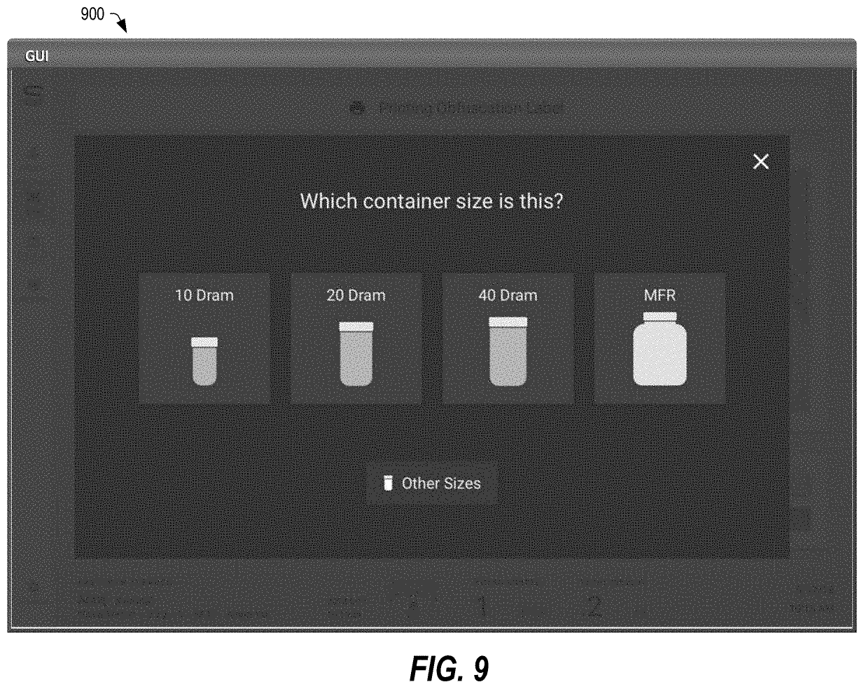

In the illustrated example, the graphical interface 800 may guide the user through a process of scanning, identifying, and classifying unsaleable pharmaceutical items into a suitable storage bin. In some embodiments, the operator may place a selected unsaleable pharmaceutical item onto the scale 108, which may cause the unsaleable pharmaceutical sorter device 102 to provide a graphical interface to prompt the user to identify a container size for the unsaleable pharmaceutical item. One possible example of such a graphical interface is described below with respect to FIG. 9.

FIG. 9 depicts a graphical interface 900 provided to the display device 104 of the unsaleable pharmaceutical sorter device 102 of FIGS. 1 and 2 after scanning a selected unsaleable pharmaceutical product and including selectable options accessible by the operator to specify a size of the container, in accordance with certain embodiments of the present disclosure. In the illustrated example, the graphical interface 900 includes four standard container size options including a first option (10 dram), a second option (20 dram), a third option (40 dram), a fourth option (manufacturer container), and an “Other Size” option. The operator may select one of the options to specify a size of the container of the unsaleable pharmaceutical item.

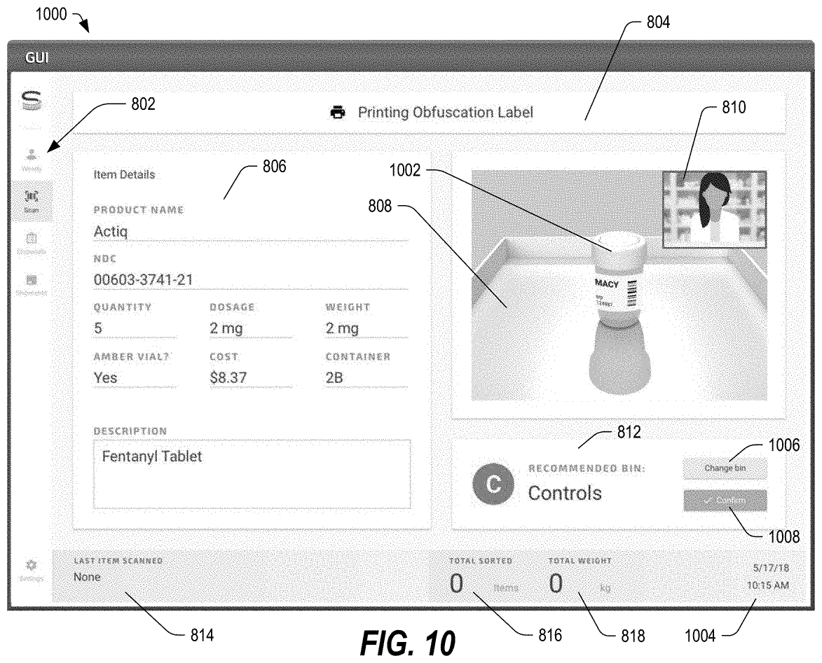

In some embodiments, while the operator is specifying the size by interacting with the graphical interface, the unsaleable pharmaceutical sorter device 102 may be configured to capture optical data (such as a barcode) associated with the container of the unsaleable pharmaceutical item and to search a local database to identify the composition of the unsaleable pharmaceutical item. Additionally, the unsaleable pharmaceutical sorter device 102 may be configured to determine a bin from a plurality of bins into which the unsaleable pharmaceutical item 102 should be placed based on the identified composition, classification rules, and optionally site-specific classification rules. One possible example of a graphical interface including the retrieved data is described below with respect to FIG. 10.

FIG. 10 depicts a graphical interface 1000 provided to the display device 104 of the unsaleable pharmaceutical sorter device 102 of FIGS. 1 and 2 after scanning, weighing, and identifying the unsaleable pharmaceutical product and after determining the container size, in accordance with certain embodiments of the present disclosure. The graphical interface 1000 may include the selectable menu options 802. Further, the graphical interface 1000 may include the item details 806, which may include a combination of the weight data and data retrieved from the local database. Further, graphical interface 1000 may include image data associated with the unsaleable pharmaceutical item 1002 within the image data from the item camera 108. In the illustrated example, the unsaleable pharmaceutical item 1002 includes a label with a barcode. Often, the label may include information identifying the contents of the bottle as well as other information. In some instances, a patient's name may be on the label.

Additionally, the graphical interface 1000 may include a recommended bin 812 for the unsaleable pharmaceutical item 1002. In this example, the recommended bin 812 is a bin labeled “Controls.” Further, with the recommendation of the “Controls” bin, the graphical interface 1000 may include a selectable button 1006 labeled “Change Bin”, which may be accessed by the operator to change the selected bin to a different selection. The graphical interface 1000 may also include a selectable button 1008 labeled “Confirm”, which may be accessed by the operator to confirm the recommended bin 812. The graphical interface 1000 also includes the last item scanned 814, a total number of items 816, and a total weight 818. Further, the graphical interface 1000 includes a date and time 1004, which can be used to date stamp data or to date and time stamp the video data captured by the item camera 108 and the operator camera 112.

It should be appreciated that, until the recommended bin 812 is confirmed by the operator by selecting the “Confirm” button 1008. Data presented within the graphical interface 1000 may be updated when the user selects the “Confirm” button 1008.

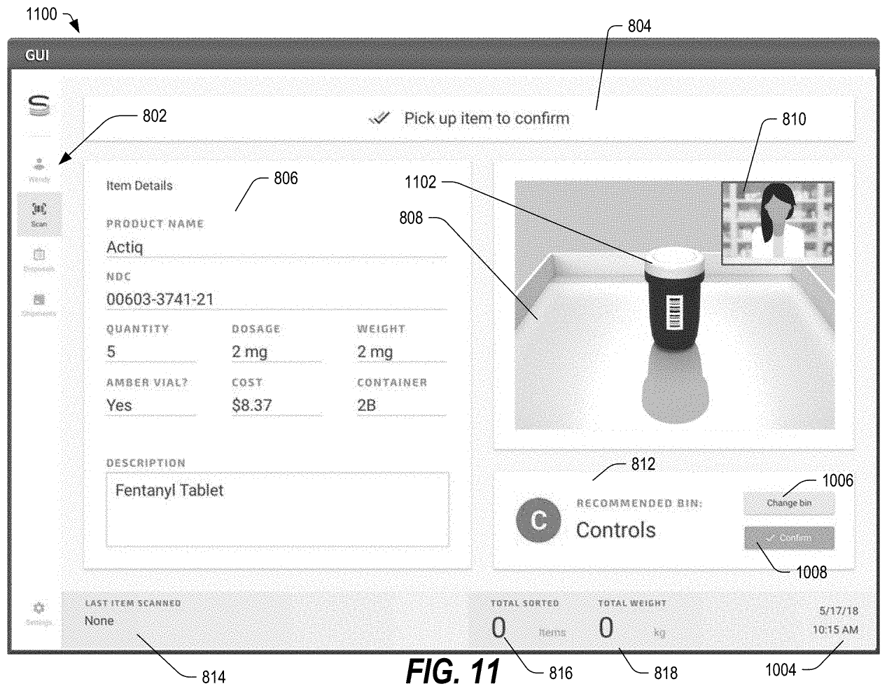

FIG. 11 depicts the graphical interface 1100 including all of the information of the graphical interface 1000 in FIG. 10 after applying a label to seal and obscure a label of the container, in accordance with certain embodiments of the present disclosure. The graphical interface 1100 further includes a description indicating “Fentanyl tablet” as the content of the unsaleable pharmaceutical item.

In this example, after selecting the “Confirm” button 1008, the unsaleable pharmaceutical sorter device 102 may print a label including a barcode and otherwise configured to seal the container and obscure an existing label on the container. The operator may apply the label to the container, such that the sealed and obscured unsaleable pharmaceutical item 1102 is shown within the optical data from the item camera 108. Other embodiments are also possible.

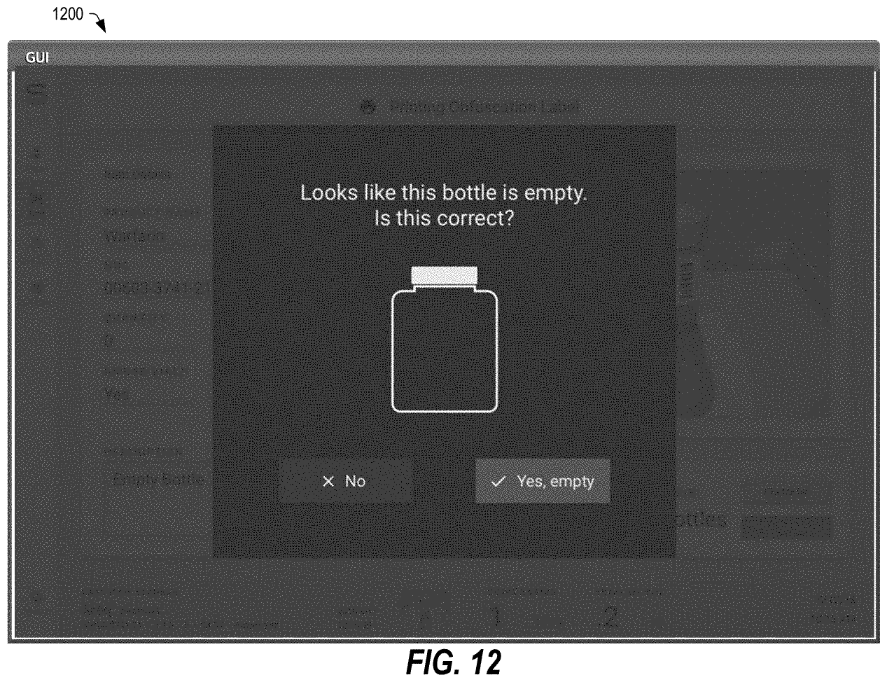

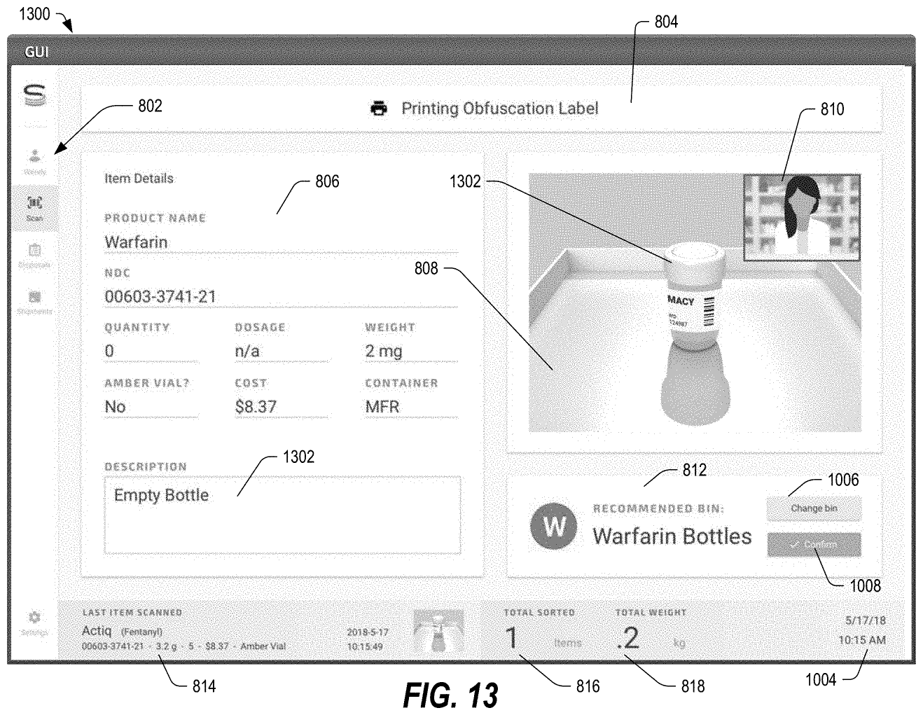

In some instances, the container itself may be considered hazardous. In the example of FIGS. 12 and 13, an unsaleable pharmaceutical item is presented that is an empty pill bottle. However, because the pill bottle was used for Warfarin, the empty bottle is still handled and disposed of as a hazardous product consistent with the disposal rules and regulations.

FIG. 12 depicts a graphical interface 1200 provided to the display device 104 of the unsaleable pharmaceutical sorter device 102 of FIGS. 1 and 2 for handling an empty container, in accordance with certain embodiments of the present disclosure. In this example, after the bottle is placed on the scale and scanned, the unsaleable pharmaceutical sorter device 102 may determine that the bottle is empty, based on the bottle size selected by the operator and the identified contents from the optical data. The graphical interface 1200 may be presented on the display device 104 to prompt the operator to confirm that the bottle is empty. The graphical interface 1200 includes a prompt and includes a “No” button and a “Yes, empty” button, which may be selected by the operator to confirm or reject the determination. Other embodiments are also possible.

FIG. 13 depicts the graphical interface 1300 provided to the display of the system of FIG. 1 after confirming that the bottle 1302 is empty using the interface 1200 of FIG. 12, in accordance with certain embodiments of the present disclosure. The graphical interface 1300 may include all of the elements of the graphical interface 1100 of FIG. 11, except that the data populating the graphical interface 1300 is different from that of the graphical interface 1100.

In a particular example, the graphical interface 1300 includes a description indicating “Empty Bottle”, which is consistent with the detected weight of the bottle and confirmed by the user's interaction with the interface 1200 in FIG. 12. Further, in this example, the last itemed scanned 814 indicates 32 grams of Actiq (Fentanyl), which represents the previously scanned item. Other embodiments are also possible.

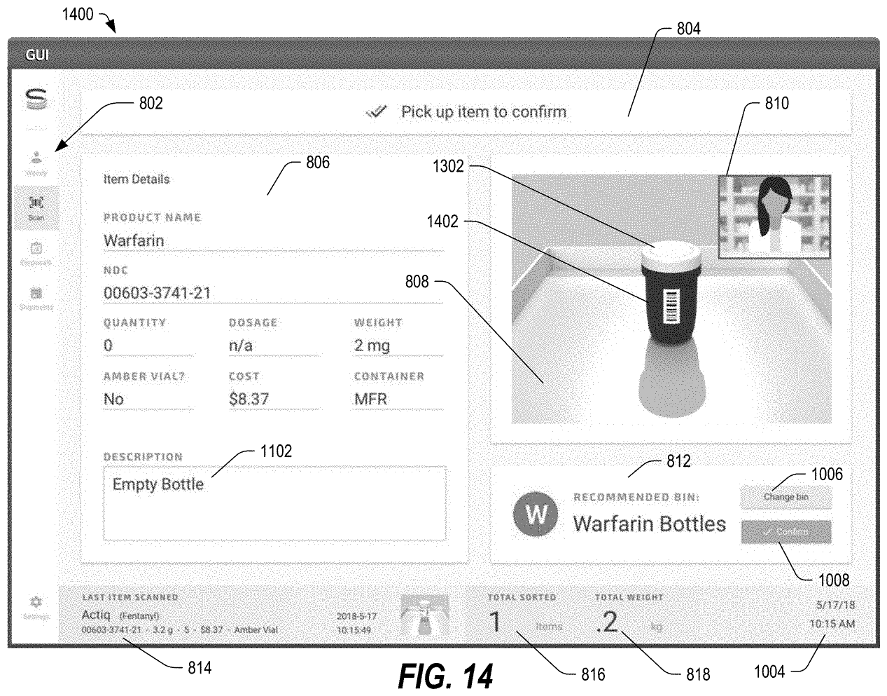

FIG. 14 depicts the graphical interface 1400 of FIG. 13 after applying a label 1402 to seal and obscure a label of the container 1302, in accordance with certain embodiments of the present disclosure. It should be appreciated that the unsaleable pharmaceutical sorter device 102 may print the label 1402 and prompt the operator to seal the container 1302 with the label 1402. The label 1402 may be provided with a barcode, which may include a unique identifier that can subsequently be used to track the packaging and disposal of the container 1302 and its contents (if any). Other embodiments are also possible.

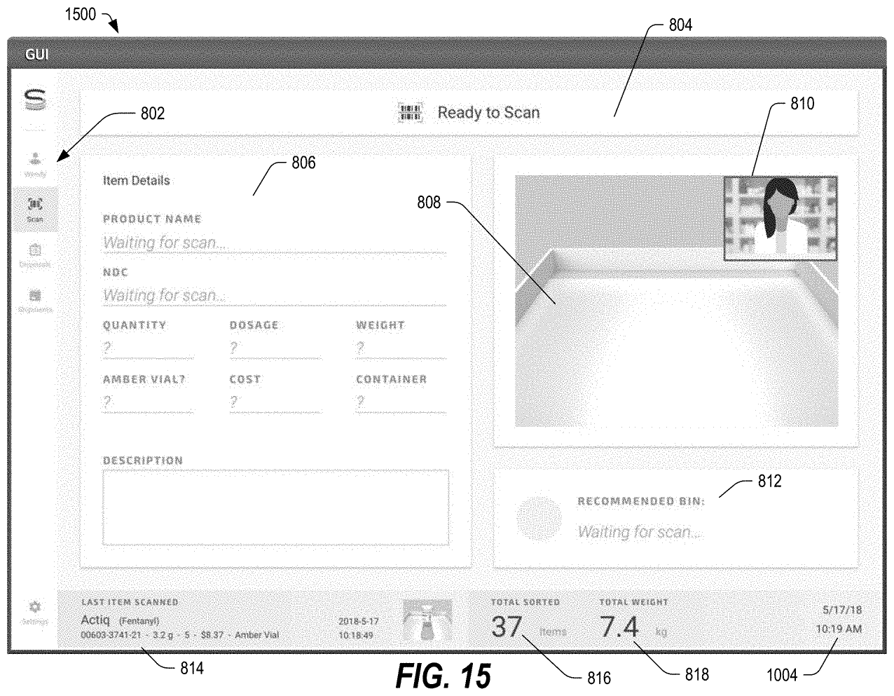

FIG. 15 depicts a graphical interface 1500 provided to the display of the system of FIG. 1 after some unsaleable pharmaceutical items have been scanned and while awaiting a next scan, in accordance with certain embodiments of the present disclosure. The graphical interface 1500 shows that 37 items have been sorted at 816 and a total weight 818 of 7.4 kg. However, the graphical interface 1500 includes a prompt indicating that the unsaleable pharmaceutical sorter device 102 is ready to scan a next item, according to the status indicator 804.

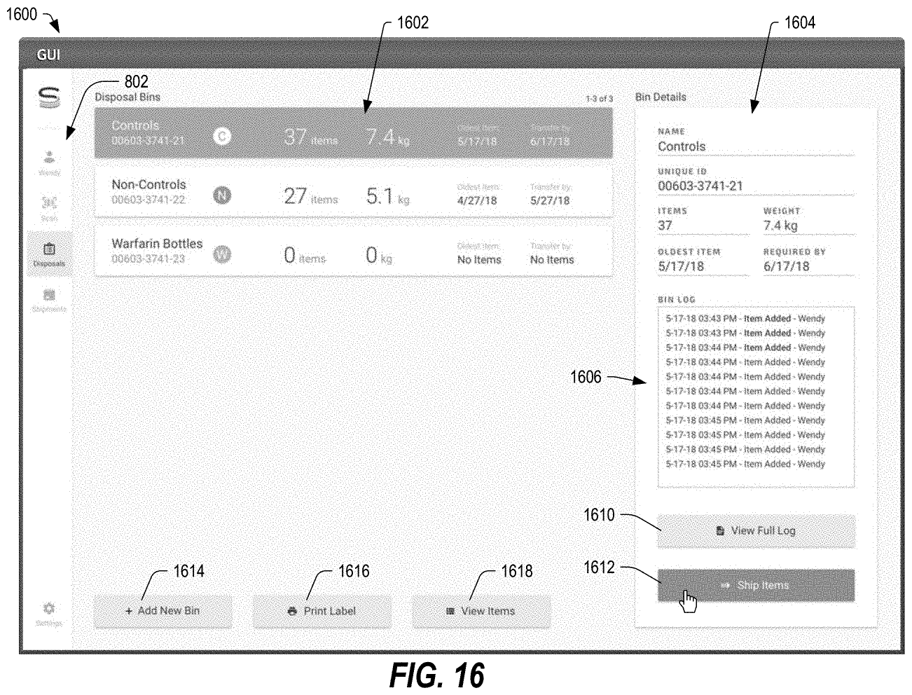

Interacting with the menu options 802 may allow an operator to review “Disposals”, which may represent previously scanned and sorted unsaleable pharmaceutical items. One possible example of an interface depicting sorted unsaleable pharmaceutical items organized into bins is described below with respect to FIG. 16.

FIG. 16 depicts a graphical interface 1600 provided to the display device 104 of the unsaleable pharmaceutical sorter device 102 of FIGS. 1 and 2 depicting a list of waste bins (“Controls”, “Non-Controls”, and “Warfarin Bottles.”) and data related to the contents of each bin, in accordance with certain embodiments of the present disclosure. The graphical interface 1600 may include a list 1602 of storage bins including a “Controls” bin, a “Non-Controls” bin, and a “Warfarin Bottles” bin. Each of the items of the list 1602 may include summary information of the content of the particular bin. Further, each item of the list 1602 can be selected by the operator to access further information. In this example, the “Controls” bin has been selected by the operator.

The graphical interface 1600 may also include a data panel 1604 including data related to the content of a selected bin, including a list of timestamps 1606 indicating when each item was added to the bin. Each item of the list of timestamps 1606 may be selectable by an operator to access details of the particular item, including image data of the unsaleable pharmaceutical item and image data of the operator. The graphical interface 1600 may further include a “View Full Log” button 1610 that can be accessed by the operator to view data corresponding to each item scanned, including the date and time of the scan, the operator, the weight, the quantity, the identified product, and so on. Additionally, the graphical interface 1600 may include a “Ship Items” button 1612 accessible by an operator to initiate a packaging and shipping process.

The graphical interface 1600 can also an “Add New Bin” button 1614, a “Print Label” button 1616, and a “View Items” button 1618. The operator may select the “Add New Bin” button 1614 to create a new storage bin and may access the “Print Label” button 1616 to print a label for the storage bin. The operator may select the “View Items” button 1618 to view items that have been scanned. Other embodiments are also possible.

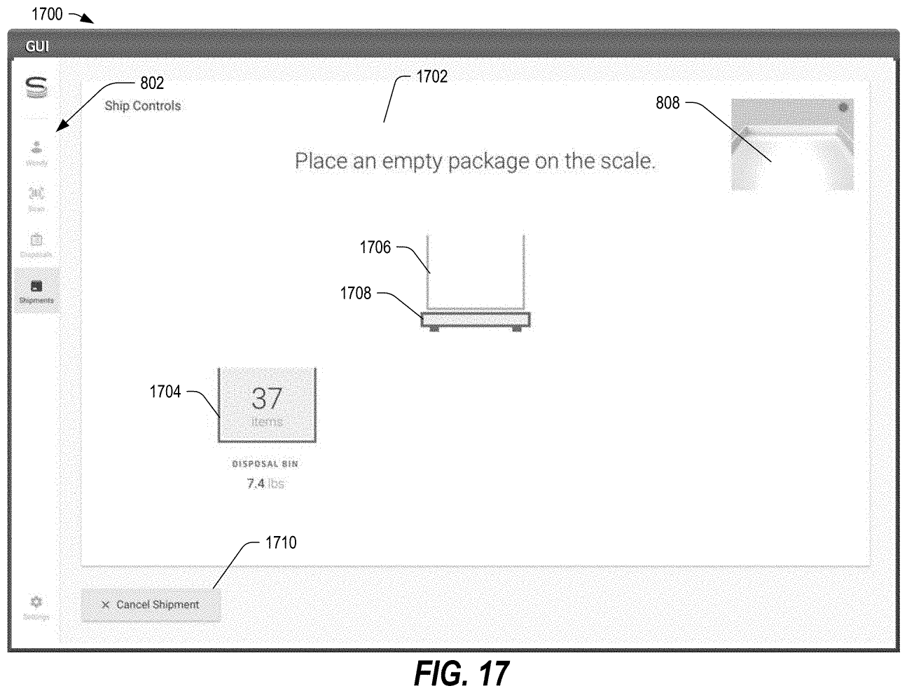

It should be appreciated that there is a difference between a bin storing multiple unsaleable pharmaceutical items and a package that is ready for shipment. In the following drawings, a method of packaging unsaleable pharmaceutical items from a storage bin into a shipping container is described in conjunction with a sequence of graphical interfaces. In response to selecting the “Shipments” option from the menu 802, a graphical interface may be presented to the display device 104 of the unsaleable pharmaceutical sorter device 102 to guide the operator through a sequence of steps and actions to create a shipping container and to transfer items from the storage bin to the shipping container to produce a package ready for shipment. One possible example of such a graphical interface is described below with respect to FIG. 17.

FIG. 17 depicts a graphical interface 1700 provided to the display device 104 of the unsaleable pharmaceutical sorter device 102 of FIG. 1 depicting a selected waste bin 1704 and an empty shipping container 1706, in accordance with certain embodiments of the present disclosure. The graphical interface 1700 may include a prompt 1702 instructing the operator to place an empty package on the scale. It should be noted that the first image 808 from the item camera 108 is presented within the graphical interface, but the operator's image from the operator camera 112 is not present. Placing an empty container onto the scale 108 may trigger the operator camera 112 to capture video.

In this example, the empty package and the scale are represented within the graphical interface 1700 as empty package 1706 and scale 1708. After placement of the shipping container or empty package 1706 on the scale 1708, the unsaleable pharmaceutical sorter device 102 may weigh the empty package to calibrate the scale 108. Further, the graphical interface 1700 may include a “Cancel Shipment” button 1710 accessible by the operator to cancel the packaging operation.

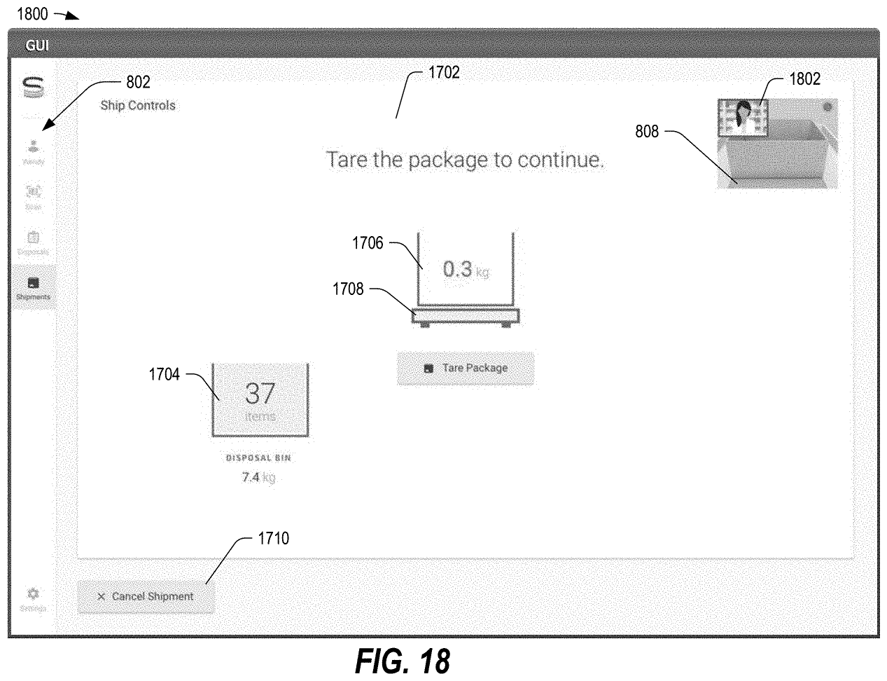

In some embodiments, an operator may assemble a box or other shipping container and place it on the scale. Subsequently, as discussed with respect to FIGS. 18-21, the operator may remove unsaleable pharmaceutical items one by one from the storage bin, rescan them, and place them into the box or shipping container. The unsaleable pharmaceutical sorter device 102 may be configured to verify the weight of each scanned item to verify that (at least, by weight) the unsaleable pharmaceutical item has not been tampered with such that the contents remain unchanged from when the item was placed in the storage bin.

FIG. 18 depicts the graphical interface 1800 including a weight of the empty shipping container 1706, in accordance with certain embodiments of the present disclosure. In this example, the shipping container 1706 remains empty, and the storage bin 1704 still reflects 37 sorted items.

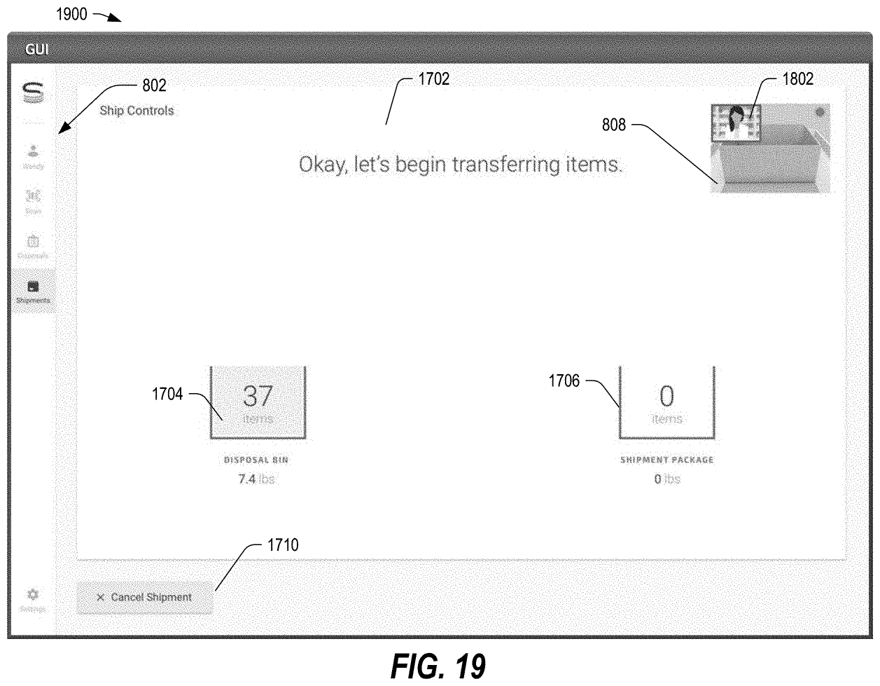

FIG. 19 depicts a graphical interface 1900 provided to the display device 104 of the unsaleable pharmaceutical sorter device 102 of FIGS. 1 and 2 configured to guide an operator through a process of transferring unsaleable pharmaceutical items from a selected bin 1704 to the shipping container 1706, showing that no items have yet been transferred, in accordance with certain embodiments of the present disclosure. The graphical interface 1900 includes a prompt for the user to begin transferring items.

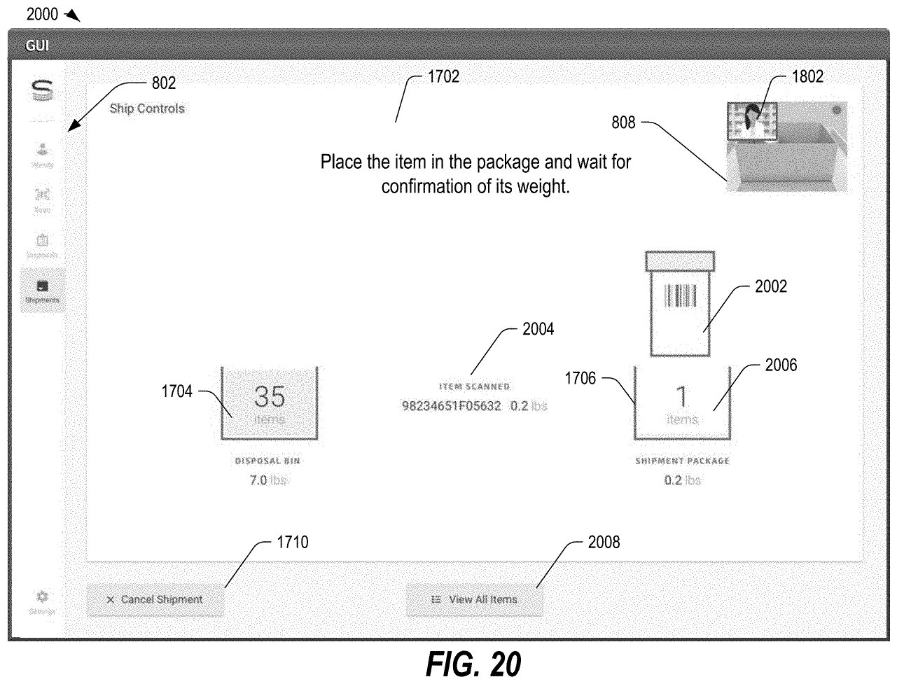

FIG. 20 depicts the graphical interface 2000 with one item transferred to the shipping container 1706, in accordance with certain embodiments of the present disclosure. In the graphical interface 2000, one item has been placed into the shipping container 1706 and the content of the storage (disposal) bin 1704 has been decremented. Further, a selected unsaleable pharmaceutical item 2002 has been removed from the content of the storage (disposal) bin 1704, and the item count has been decremented again. Additionally, the weight of the storage (disposal) bin 1704 has decreased in correspondence with the weight of the unsaleable pharmaceutical items that have been removed. As the items are scanned, the graphical interface 2000 prompts the operator to “Place the item in the package and wait for confirmation of its weight.” Thus, the unsaleable pharmaceutical sorter device 102 is configured to confirm the current weight determined during the current scan operation relative to the previous scan/weigh operation performed prior to storage in the bin 1704.

The previously assigned identifier and weight of the selected unsaleable pharmaceutical item 2002 are retrieved from memory, in response to the scan operation, and can be displayed in the graphical interface 2000, as generally indicated at 2004. The current content of the shipping container 1706 is generally indicated at 2006, showing 1 item prior to the placement of the currently scanned item 2002 into the shipping container.

The graphical interface 2000, once an item has been scanned, may show a “View All Items” button 2008, which can be accessed by the operator to view a list of scanned waste items. Other embodiments are also possible.

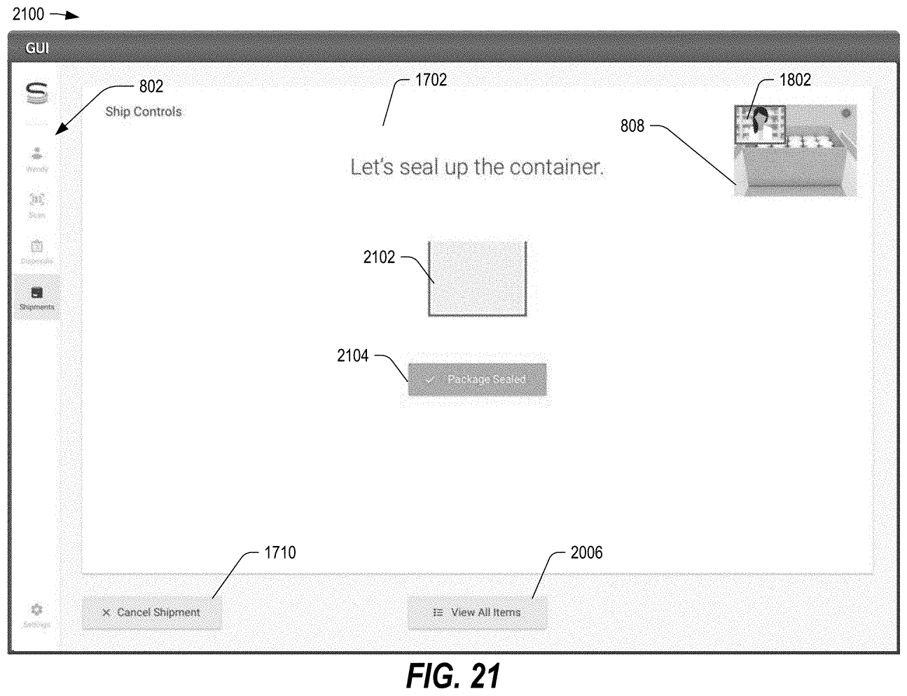

FIG. 21 depicts a graphical interface 2100 provided to the display device 104 of the unsaleable pharmaceutical sorter device 102 of FIGS. 1 and 2 configured to guide the operator to seal the shipping container, in accordance with certain embodiments of the present disclosure. Once all of the items have been transferred from the storage (disposal) bin 1702 to the shipping container 1706, the graphical interface 2100 may present a prompt 1702 that says “Let's seal up the container.” The graphical interface 2100 may further include a graphical representation of the shipping container 2102 and a “Package Sealed” button 2104 that can be selected by the operator once the shipping container 2102 has been sealed.

In some embodiments, prior to sealing of the container, the unsaleable pharmaceutical sorter device 102 may print a manifest for inclusion in the shipping container 2102. Further, in some embodiments, sealing the shipping container may include taping a box closed for shipping and attaching a shipping label and optionally a manifest to the shipping container. Other embodiments are also possible.

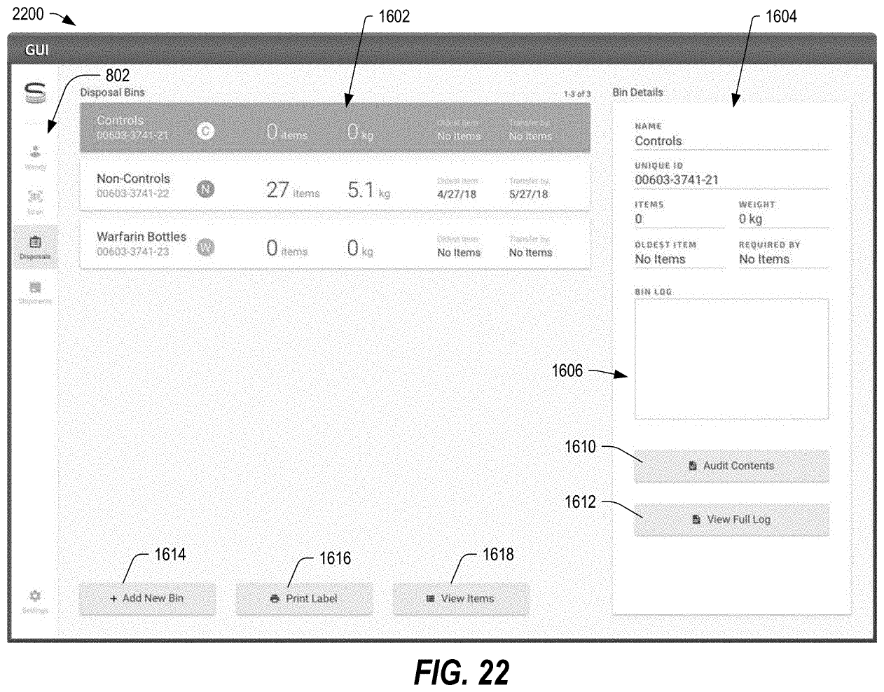

FIG. 22 depicts the graphical interface 2200, which may be a version of the graphical interface 1600 of FIG. 16 with the “Controls” bin reset to reflect zero items, in accordance with certain embodiments of the present disclosure. In an example, once the storage (disposal) bin is empty and the shipping container is sealed, the contents of the storage (disposal) bin may be reset to zero.

In the example of FIG. 22, selection of the “Controls” bin from the list 1602 displays the absence of waste items. Further, the log of waste items for the “Controls” bin is cleared, at 1604. Subsequent scans of waste items that are categorized for storage in the “Controls” bin will update the log 1604. Other embodiments are also possible.

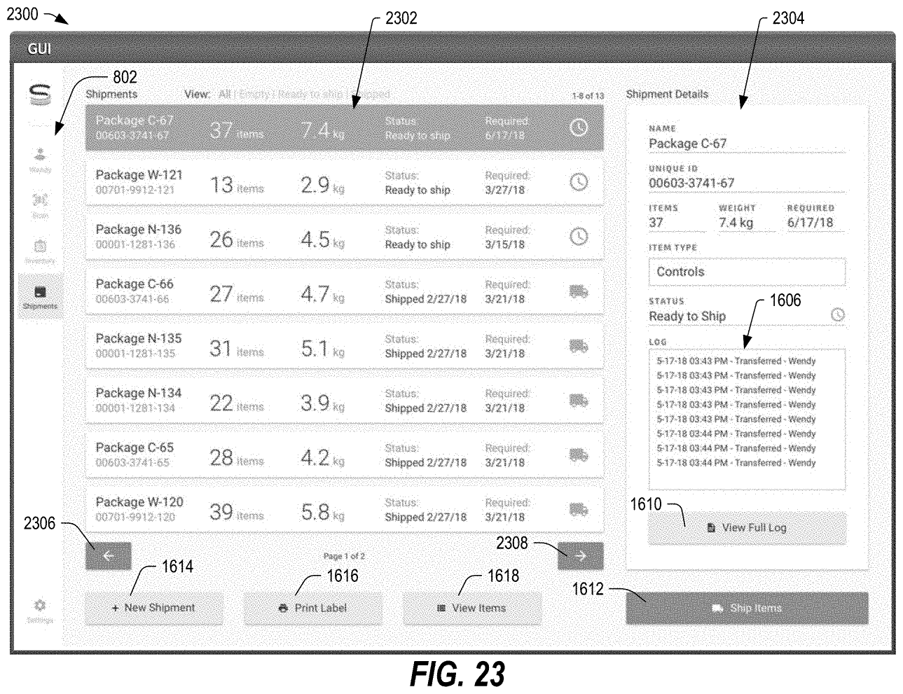

FIG. 23 depicts a graphical interface 2300 that can be provided to the display device 104 of the unsaleable pharmaceutical sorter device 102 of FIGS. 1 and 2 including a list of shipping containers or packages 2302, in accordance with certain embodiments of the present disclosure. In the illustrated example, each container or package 2302 in the list can be a selectable element that, when selected, may cause the graphical interface 2300 to display shipment details 1604 related to the selected package. Further, the graphical interface 2300 may include a left arrow 2306 and a right arrow 2308 accessible by the operator to scroll through the list of shipment containers or packages 2302 to view other package data. Other embodiments are also possible.

In conjunction with the systems, methods, and devices described above with respect to FIGS. 1-23, an unsaleable-pharmaceutical sorter device is disclosed that is configured to concurrently capture optical data associated with a unsaleable pharmaceutical item and an operator of the device. Further, the unsaleable pharmaceutical sorter device may be configured to identify a unsaleable pharmaceutical item based on the optical data, determine a bin into which the unsaleable pharmaceutical item is to be sorted, determine a weight of the item, print a label configured to adhere to the waste item to seal the container and to obscure an existing label of the container, and create a record for the waste item in a memory. The record may include the optical data, retrieved and determined information about the waste item, a unique identifier, and a bin assignment for storage of the waste item in a selected bin of a plurality of bins.

Subsequently, when the storage bin is full or ready for disposal, the unsaleable pharmaceutical sorter device may be configured to guide the operator to create a package or shipping container and to load the shipping container with the items from the storage bin. Each item may be rescanned and the weight verified as the item is placed into the shipping container to ensure transfer of the same items and quantity. The unsaleable pharmaceutical sorter device may print a manifest and a shipping label for the container. Further, the records in the memory may be updated to indicate each item that has been packaged and shipped. Other embodiments are also possible.

Although the present invention has been described with reference to preferred embodiments, workers skilled in the art will recognize that changes may be made in form and detail without departing from the scope of the invention.

What is claimed is:

1. A device comprises:
at least one camera configured to capture first optical data of a unsaleable pharmaceutical item and second optical data of an operator;
a display device; and
a processor coupled to the at least one camera and the display device, the processor configured to:
provide a graphical interface to the display device, the graphical interface including information to guide the operator through a process of sorting items for disposal including the unsaleable pharmaceutical item, the graphical interface including the first optical data and the second optical data.

2. The device of claim 1, wherein the processor is configured to determine composition information corresponding to the unsaleable pharmaceutical item based on the first optical data.

3. The device of claim 2, further comprising a scale coupled to the processor and configured to determine a weight of the unsaleable pharmaceutical item.

4. The device of claim 3, wherein the processor is configured to determine a quantity of pills associated with the unsaleable pharmaceutical item based in part on the weight.

5. The device of claim 2, wherein the processor is configured to determine a disposal bin of a plurality of disposal bins based on the composition information and a set of disposal rules.

6. The device of claim 1, further comprising a printer coupled to the processor and configured to print a label to be applied to a container of the unsaleable pharmaceutical item to seal the container and to obscure any existing label of the container.

7. The device of claim 1, further including a memory coupled to the processor and configured to store data related to the unsaleable pharmaceutical item including the first optical data, the second optical data, data determined based on the first optical data, weight data, and a timestamp.

8. The device of claim 7, wherein the processor is further configured to:
provide a second graphical interface to guide the operator through a process of packaging a plurality of unsaleable pharmaceutical items including the unsaleable pharmaceutical item from a disposal bin into a shipping container; and
the processor configured to receive data as each of the plurality of unsaleable pharmaceutical items is transferred for verification.

9. A device comprising:
at least one optical sensor configured to capture first optical data associated with an unsaleable pharmaceutical product and to capture second optical data associated with an operator;
a display device; and
a processor coupled to the at least one optical sensor and the display device, the processor configured to:
determine a disposal bin from a plurality of disposal bins based on the first optical data and a set of disposal rules; and
provide a graphical interface including a bin identifier, the first optical data, and the second optical data to the display device.

10. The device of claim 9, further comprising a scale configured to determine a weight associated with the unsaleable pharmaceutical product.

11. The device of claim 10, further comprising:
a memory coupled to the processor; and
wherein the processor is configured to store a record including data corresponding to the unsaleable pharmaceutical product, the first optical data, the second optical data, the weight, a timestamp, and the bin identifier.

12. The device of claim 10, wherein the processor is configured to determine a quantity of pills associated with the unsaleable pharmaceutical item based in part on the weight.

13. The device of claim 9, further comprising a printer coupled to the processor and configured to print a label to be applied to a container of the unsaleable pharmaceutical item to seal the container and to obscure any existing label of the container.

14. The device of claim 13, wherein the processor is further configured to:
provide a second graphical interface to guide the operator through a process of packaging a plurality of unsaleable pharmaceutical items including the unsaleable pharmaceutical item from the disposal bin into a shipping container; and
the processor configured to receive data as each of the plurality of unsaleable pharmaceutical items is transferred for verification.

15. The device of claim 14, wherein the processor causes the printer to print a shipping manifest and a shipping label for the shipping container.

16. A method comprising:
receiving first optical data of a unsaleable pharmaceutical item from at least one camera of a sorter device;
receiving second optical data of an operator from the at least one camera; and
providing a graphical interface to a display device of the sorter device, the graphical interface including information to guide the operator through a process of sorting unsaleable pharmaceutical items for disposal, the graphical interface including the first optical data and the second optical data.

17. The method of claim 16, further comprising:
determining composition information corresponding to a unsaleable pharmaceutical item based on the first optical data;
automatically selecting a disposal bin from a plurality of bins based on the composition information and a set of rules; and
providing an identifier associated with the selected disposal bin within the graphical interface.

18. The method of claim 16, further comprising determining a weight of the at least one unsaleable pharmaceutical item using a scale.

19. The method of claim 16, further comprising printing an adhesive label for applying to a container of the unsaleable pharmaceutical product to seal the container and to obscure an existing label on the container.

20. The method of claim 19, further comprising:
providing a graphical interface including prompts to guide an operator through a process of packaging a plurality of unsaleable pharmaceutical items from the disposal bin into a shipping container;
printing a manifest associated with the shipping container; and
printing a label to be applied to the shipping container.
